(19) United States
(12) Patent Application Publication
(10) Pub. No.: US 2011/0282315 A1
(43) Pub. Date: Nov. 17, 2011

(54) PACKAGE FOLDING OF DISPOSABLE SANITARY ARTICLES HAVING BELTS

(75) Inventors: Maria Gustin Bergström, Halso (SE); Magnus Bergendahl, Molnlycke (SE); Lars Melin, Goteborg (SE)

(73) Assignee: SCA HYGIENE PRODUCTS AB, Goteborg (SE)

(21) Appl. No.: 13/130,620

(22) PCT Filed: Nov. 28, 2008
(86) PCT No.: PCT/SE2008/051372

§ 371 Date: Jul. 22, 2011

(51) Int. Cl. A61F 13/84; B65B 63/04

(52) U.S. Cl. 604/385.201; 53/429

(57) ABSTRACT

A method of package folding a disposable sanitary article and article thereof having a front part, a rear part, a crotch part therebetween, and a belt including two parts. Each of the two belt parts can be extended in a cross-direction outwardly from the respective outer end portion of each side portion of the rear part. The belt is arranged on the rear part with the belt parts overlapping each other. Fastening elements for releasably fastening the front part to the belt are arranged on each side portion of the front part. The article is folded so that the folded article attains a rectangular shape and a desired size. As a first step in the package folding, the front and rear parts are folded towards each other so that the fastening elements on the front part are attached to one of the overlapping belt parts arranged on the rear part.

### CROSS-REFERENCE TO PRIOR APPLICATION

This application is a §371 National Stage Application of PCT International Application No. PCT/SE2008/051372 filed Nov. 28, 2008.

### FIELD OF THE INVENTION

The present disclosure relates to a method of package folding a disposable sanitary article having a front part, a rear part and a crotch part therebetween, and a belt including two parts. Each of the two parts can be extended in a cross-direction outwardly from the respective outer end portion of each side portion of the rear part. The belt is arranged on the rear part with the belt parts overlapping each other. The means for releasably fastening the front part to the belt are arranged on each side portion of the front part. The method includes folding the article so that the folded article attains a rectangular shape and a desired size.

### BACKGROUND

Nowadays, hooks and loops fasteners are often used on disposable sanitary articles, such as incontinence protectors and diapers. It is also often the case that hooks fastener elements that attach to certain types of nonwoven (NW) materials are used in such articles and those certain types of NW are used for, for example, an outer layer in a composite back sheet of a diaper or the outside layer of a belt in an incontinence protector. A problem that might arise when package folding such articles is that there is a risk that hooks in the hooks fastener element attach to other NW materials, for example the top sheet of an incontinence protector or a diaper, instead of the certain types of NW the hooks fastener element is constructed to co-operate with. If such an attachment occurs there is a risk that either hooks in the hooks fastener element or the NW material in question are/is damaged or even result in breakage of the hooks or the NW material.

A problem observed for package folded incontinence protectors having belts is that users have problem in understanding how to apply such a protector.

### SUMMARY

The present disclosure eliminates the above mentioned risk and reduces the above mentioned problem.

A first aspect includes a method of package folding a disposable sanitary article having a front part, a rear part and a crotch part therebetween, and a belt including two parts. Each of the two belt parts can be extended in a cross-direction outwardly from the respective outer end portion of each side portion of the rear part. The belt is arranged on the rear part with the belt parts overlapping each other. Means for releasably fastening the front part to the belt are arranged on each side portion of the front part. The method includes folding the article so that the folded article attains a rectangular shape and a desired size, wherein the front and rear parts are folded towards each other so that the fastening means on the front part are attached to one of the overlapping belt parts arranged on the rear part, as a first step in the package folding of the article. By attaching the fastening means on the front part to the belt before folding-in the side portions of the article, the risk that the fastening means become attached to the top sheet is eliminated. Furthermore, the attachment of the fastening means on the front part to the belt indicates to the user unfolding the package folded article, how these fastening means should be attached and helps the user to understand how the article should be applied, especially that the fastening means on the front part of the article should be attached to the outside of the belt.

In a particular embodiment, the article has an hour-glass shape, and opposite side portions of the once folded article are folded about longitudinal folding lines so that the folded article attains a rectangular shape. The article can be folded about a cross-directional line before or after the step of folding opposite side portions of the once folded article.

A second aspect includes a package folded disposable sanitary article having a front part, a rear part and a crotch part therebetween, and a belt including two parts. Each of the two belt parts can be extended in a cross-direction outwardly from the outer end portion of each side portion of the rear part. The belt is arranged on the rear part with the belt parts overlapping each other. Means for releasably fastening the front part to the belt are arranged on each side portion of the front part. The article is folded so that the folded article attains a rectangular shape and a desired size, wherein the front and rear parts are folded towards each other so that the fastening means on the front part are attached to one of the overlapping belt parts arranged on the rear part.

In a particular embodiment, the article has an hour-glass shape in unfolded state, and opposite side portions of the once folded article are folded about longitudinal folding lines so that the folded article has attained a rectangular shape.

### BRIEF DESCRIPTION OF THE DRAWINGS

Embodiments of the invention will now be described with reference to the enclosed figures, of which,

FIG. 1 schematically discloses an embodiment of an incontinence protector in a plan view and with the elastics of the protector in an extended state, and

FIGS. 2A-E schematically illustrates the different steps in a method of package folding the disposable incontinence protector in FIG. 1.

### DETAILED DESCRIPTION OF PREFERRED EMBODIMENTS

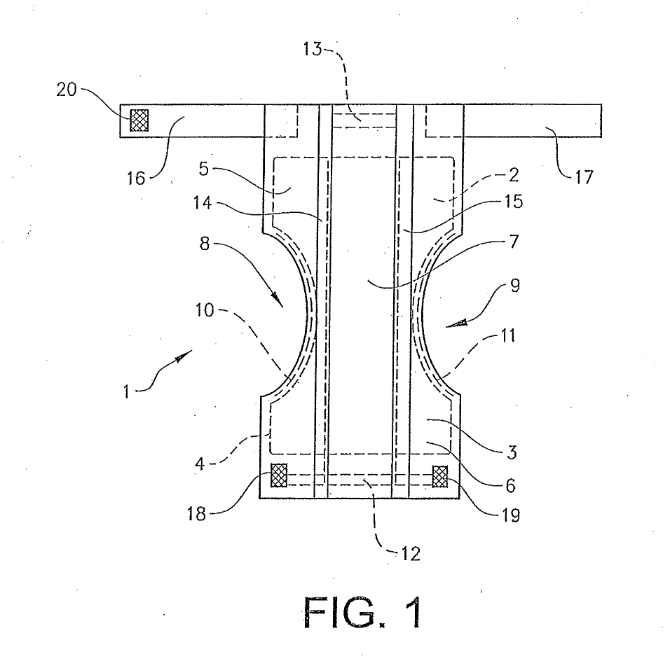

In FIG. 1, an incontinence protector 1 is schematically disclosed in a plan view from above. The protector includes an absorbent body 2 enclosed between a top sheet 3 and a back sheet 4. The protector includes a rear part 5, a front part 6 and a crotch part 7 extending therebetween. The protector also includes leg openings 8,9 and leg elastics 10,11 and also waist elastics 12,13 in the front and rear part, respectively. Barrier flaps 14,15 for preventing sidewise leakage of liquid from the top sheet 3 extend in a longitudinal direction of the protector 1 and have elastic elements along the longitudinal edges thereof turned towards each other. By the presence of leg openings an hour-glass shape is given to the protector.

The protector 1 also includes belt parts 16, 17 which can be extended out laterally, i.e. in a cross-direction in relation to a longitudinal direction extending from the rear part to the front part, from the rear part 5 of the protector, as illustrated in FIG. 1. Furthermore, fastener elements 18, 19 containing hooks are disposed in the outer corner portions of the front part 6 of the protector on the inside thereof, i.e. affixed to the top sheet 3. A fastener element 20 containing hooks is affixed to the inside of the outer end of one of the belt parts 16, 17, in the present case to belt part 16. These fastener elements are to be attached to the outer side of the belt when the protector is applied to a user. For this purpose the outer side of belt part 17 includes loops elements for adjustably attaching the hooks element 20 thereto. The belt parts 16,17 can for example include a nonwoven of the type to which hooks element attach or have loops element affixed to the outsides of the belt parts extending along at least a portion of the belt parts.

In FIG. 1, the protector 1 is shown with all elastic elements in an extended state.

The materials used for the components of the protector can be any of those that are known to be used for similar components in known incontinence protectors or diapers.

The protector 1 is to be applied by placing the rear part 5 of protector 1 at the back of the user and putting the belt parts 16, 17 around the waist and attach the fastener element 20 to the outer side of belt part 17. The applied belt is then, if necessary, adjusted in the circumferential direction so that the front part 6 of the protector 1 is located between the legs of the user. Thereafter, the front part is gripped by the user and moved upwards. The waist portion of the front part is then held in a stretched condition and the hooks fastener elements 18, 19 are attached to the outside of the applied belt.

A problem with known package folded protectors is that the hooks fastener elements 18, 19 are folded-in onto the top sheet. Thereby there is a risk that the hooks in these elements 18, 19 will attach to the top sheet which includes nonwoven material of a type not intended to co-operate with a hooks fastener element. This can lead to breakage of hooks in the fastener elements or of the top sheet.

It has also been shown that new users have problem in understanding how to apply the protector 1. In many cases they try to apply the protector in the same way as an all-in-one diaper. It is believed that this is partly due to the fact that in known package folded protectors the belt parts are folded-in over the rear part and not easily recognizable as belt parts to the user.

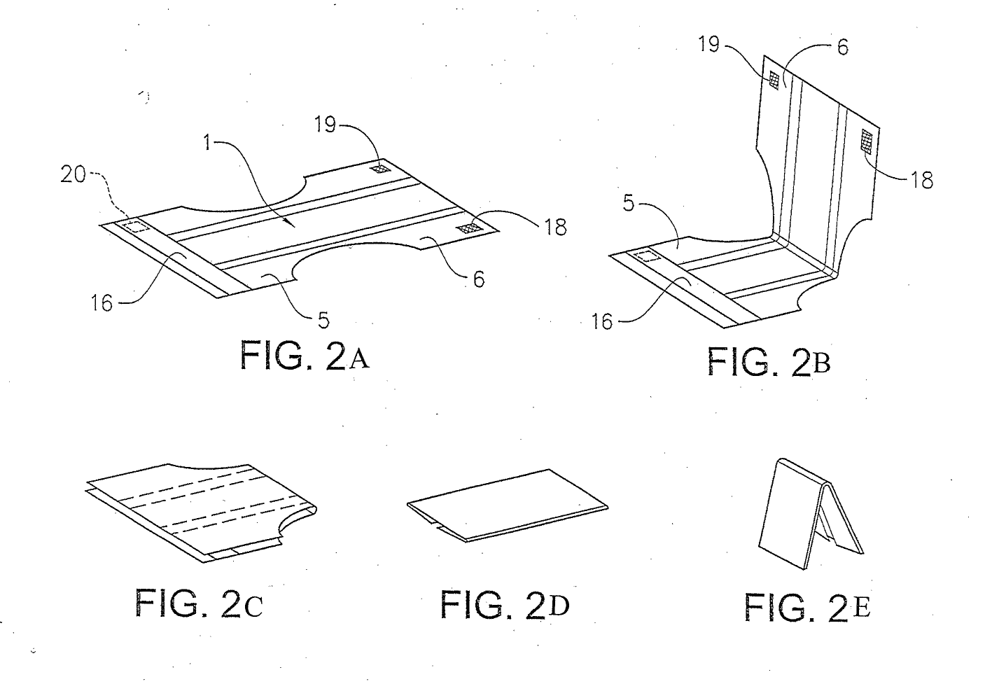

These problems are solved or at least reduced by the package folding of the protectors illustrated in FIGS. 2A-E.

In FIGS. 2A-E, the different steps in the package folding of a protector 1 is schematically illustrated.

FIG. 2A shows the protector 1 after manufacturing thereof. During manufacture, the belt parts 16, 17 are applied over the rear part 5 overlapping each other as shown in FIG. 2A. The belt part 16 then totally covers belt part 17 and the hooks fastener element 20 is attached to the outside of belt part 17.

In the next step shown in FIGS. 2B and 2C, the front and rear parts 5, 6 are folded-in towards each other about a cross-directional folding line in the middle of the crotch part. In FIG. 2B, about half of this folding-in step has been performed and in FIG. 2C this folding step has been totally performed. The cross-directional folding line is chosen so that the hooks fastener elements 18, 19 will be pressed against the outside of the belt part 16 which for the protector 1 means that the outer free end edges of rear and front parts 5, 6 will lie flush to each other, as is evident from FIG. 2C.

In the next folding step shown in FIG. 2D, the once folded protector 1 is folded to a rectangular shape by folding the lateral sides of the once folded protector in towards the lower side of the once folded protector about folding lines schematically indicated in FIG. 2C by interrupted lines. By the term “lower” is meant as seen by a viewer of FIGS. 2A-E.

Finally, the twice folded protector 1 is again folded, about a cross-directional line, as is shown in FIG. 2E.

Evidently, the step of folding the lateral sides of the once folded protector can as well be performed in the opposite direction as shown in FIG. 2D, i.e. towards the upper side of the once folded protector. In such a case it is preferred, but not necessary, that the folding step illustrated in FIG. 2E is performed in the opposite direction so that the lateral side portions folded in towards the upper side will not be present on the outside of the package-folded protector.

Thereafter the package folded protector 1 is fed to a stacker and bag filler.

By the folding step illustrated in FIGS. 2B and 2C, the hooks fastener elements 18, 19 are attached to the outside of belt part 16. Thereby, the risk that the fastener elements 18,19 will be unintentionally attached to the top sheet resulting in damage to the hooks or the top sheet, is eliminated.

Moreover, when a protector 1 is taken out of a package, unfolding thereof will lead to the once folded protector shown in FIG. 2C. The pants-like configuration of the protector 1 in the once folded state corresponds essentially to the configuration the protector 1 shall have after being applied correctly on a user. A user unfolding the protector 1 is thereby given an indication of how to apply the protector in order to obtain a pants-like configuration. Since the user has to release the hooks fastener elements 18, 19 from the belt part 16 in order to unfold the protector to the state shown in FIG. 2A, a user will at least learn that elements 18, 19 attach well to the outside of belt part 16. This knowledge would make it easier for a new user to understand how the protector 1 shall be applied.

In the illustrated embodiment of the package folded protector 1, the step according to FIG. 2D of folding the lateral sides of the once folded protector to obtain a rectangular shape of the protector is made before the final step of folding the protector to a desired rectangular size. However, the folding steps illustrated in FIGS. 2D and 2E can change place, i.e. the folding of the protector about a cross-directional line to obtain a desired size, illustrated in FIG. 2E, can be made before a final folding step of folding in the lateral sides of a twice folded protector to obtain a rectangular shape. It is of course also possible to perform further folding steps after the steps shown in FIGS. 2A-2E if a smaller size of the package folded protector is desired.

Devices for performing the different folding steps are known per se and need not be described in order to facilitate understanding of the present invention. It is enough to point out that the protector 1 is held with its elastic elements extended during manufacture and until the final folding step has been performed. To advantage this is accomplished by sub-pressure acting on the back sheet of the protector or by clamping devices or the like.

The embodiments described can be modified without leaving the scope of invention. For example, the protector can have another shape, e.g. be without leg openings, and be without barrier flaps. The belt parts need not be made in one piece of material but can have elastic portions. The fastener elements need not be hooks and loops elements, even if this is preferred, but can for example be adhesive patches on the front part and plastic areas on the belt parts to which the adhesive patches can be releasably attached. The belt parts can have other dimensions and be attached to the rear part in other ways. The belt parts need not be separate elements but can be connected to each other by an intermediate part. A step of folding in the belt parts over the rear part so that portions of the different parts overlap each other may thus be part of the manufacturing process. If the article attains a desired size after the folding in of the lateral sides of the once folded article to obtain a rectangular shape the further folding steps in the described embodiment are deleted. The scope of protection shall therefore only be restricted by the content of the enclosed patent claims.

1. A method of package folding a disposable sanitary article having a front part, a rear part, a crotch part therebetween, and a belt including two parts, wherein each belt part can be extended in a cross-direction outwardly from the respective outer end portion of each side portion of the rear part, wherein the belt is arranged on the rear part with the belt parts overlapping each other, and wherein fastening elements for releasably fastening the front part to the belt are arranged on each side portion of the front part,
said method includes folding the article so that the folded article attains a rectangular shape and a desired size, which includes folding the front and rear parts towards each other so that said fastening elements on the front part are attached to one of the overlapping belt parts arranged on the rear part, as a first step in the package folding of the article.

2. The method according to claim 1, wherein the article has an hour-glass shape and opposite side portions of the once folded article are folded about longitudinal folding lines so that the folded article attains a rectangular shape.

3. The method according to claim 2, wherein the article is folded about a cross-directional line before or after the step of folding opposite side portions of the once folded article.

4. A package folded disposable sanitary article having a front part, a rear part, and a crotch part therebetween, and a belt including two parts,
wherein each belt part can be extended in a cross-direction outwardly from the outer end portion of each side portion of the rear part, the belt is arranged on the rear part with the belt parts overlapping each other, and fastening elements for releasably fastening the front part to the belt are arranged on each side portion of the front part, and
wherein the article is folded so that the folded article attains a rectangular shape and a desired size, wherein the front and rear parts are folded towards each other so that said fastening elements on the front part are attached to one of the overlapping belt parts arranged on the rear part.

5. A package folded disposable sanitary article according to claim 4 having an hour-glass shape in unfolded state, wherein opposite side portions of the once folded article are folded about longitudinal folding lines so that the folded article has attained a rectangular shape.
